(19) United States
(12) Patent Application Publication
(10) Pub. No.: US 2024/0385181 A1
(43) Pub. Date: Nov. 21, 2024

(54) SAMPLE PROCESSING APPARATUS AND HOUSING CONTAINER

(71) Applicants: Canon Kabushiki Kaisha, Tokyo (JP); CANON MEDICAL SYSTEMS CORPORATION, Otawara-shi (JP)

(72) Inventor: Shinya SUGIYAMA, Nasushiobara (JP)

(73) Assignees: Canon Kabushiki Kaisha, Tokyo (JP); CANON MEDICAL SYSTEMS CORPORATION, Otawara-shi (JP)

(21) Appl. No.: 18/664,391

(22) Filed: May 15, 2024

(30) Foreign Application Priority Data
May 16, 2023 (JP) 2023-080641

(51) Int. Cl. G01N 33/52; G06T 7/90

(52) U.S. Cl. CPC G01N 33/521; G06T 7/90

(57) ABSTRACT

A sample processing apparatus according to an embodiment includes a closed-system flow channel, a housing container, and a detection member. The closed-system flow channel is a flow channel configured to process a liquid containing a sample. The housing container houses the flow channel. The detection member is a member provided inside the housing container and configured to detect a liquid present in a space inside the housing container.

### CROSS-REFERENCE TO RELATED APPLICATIONS

This application is based upon and claims the benefit of priority from Japanese Patent Application No. 2023-080641, filed on May 16, 2023, the entire contents of which are incorporated herein by reference.

### FIELD

Embodiments described herein relate generally to a sample processing apparatus and a housing container.

### BACKGROUND

Conventionally, closed-system devices such as cell culture devices including closed-system flow channels and cell culture devices including closed-system containers have been known. In such closed-system devices, samples having infectiousness may be subjected to processing. In such cases, it is important to check the presence or absence of leakage of liquid (hereinafter also referred to as “liquid leakage”) from closed-system devices from the viewpoint of infection prevention. It is also important to check the presence or absence of liquid leakage in order to prevent the mixing of foreign matter from outside closed-system devices.

### BRIEF DESCRIPTION OF THE DRAWINGS

FIG. 1 is a diagram for illustrating an example of a cell culture apparatus according to a first embodiment;

FIG. 2 is a diagram for illustrating an example of a cell culture apparatus according to a second embodiment;

FIG. 3 is a diagram for illustrating an example of a cell culture apparatus according to a third embodiment;

FIG. 4 is a diagram for illustrating an example of liquid detection processing according to a modification;

FIG. 5 is a diagram illustrating an example of gap detection processing according to the modification;

FIG. 6 is a diagram illustrating an example of a cell culture apparatus in a different form from the embodiment;

FIG. 7 is a diagram for illustrating an example of a cell culture apparatus different from the embodiment including closed-system flow channels; and

FIG. 8 is a diagram for illustrating an example of a cell culture apparatus different from the embodiment including closed-system flow channels.

### DETAILED DESCRIPTION

A sample processing apparatus according to the present embodiment includes a closed-system flow channel, a housing container, and a detection member. The closed-system flow channel is a flow channel configured to process a liquid containing a sample. The housing container houses the flow channel. The detection member is a member provided inside the housing container and configured to detect a liquid present in a space inside the housing container.

The following describes embodiments of the sample processing apparatus and the housing container in detail with reference to the accompanying drawings. The sample processing apparatus and the housing container according to the present application are not limited by the following embodiments. The embodiments can be combined with other embodiments and conventional technologies to the extent that their contents produce no contradictions. In the following description, similar components are denoted by common symbols, and duplicated descriptions are omitted.

### First Embodiment

A cell culture apparatus according to the present embodiment is an example of a sample processing apparatus. The cell culture apparatus according to the present embodiment is a double structure container including a housing container housing a closed-system flow channel. The housing container includes a detection member detecting liquid leakage from the closed-system flow channel thereinside.

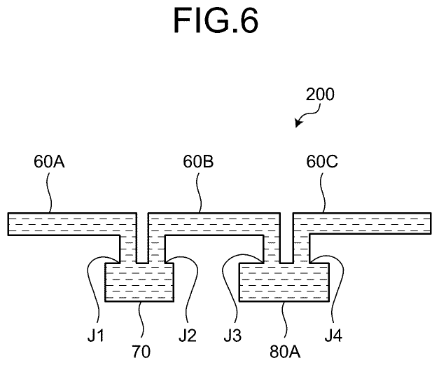

The following first describes an example of a general cell culture apparatus including the closed-system flow channel. FIG. 6 to FIG. 8 are diagrams for illustrating the example of the cell culture apparatus including the closed-system flow channel.

This cell culture apparatus 200 illustrated in FIG. 6 includes closed-system flow channels 60A to 60C, a reagent container 70, and a culture container 80A. The closed-system flow channels 60A to 60C are pipes configured to feed, for example, reagents such as culture media for use in cell culture, cell suspensions containing target cells to be cultured, or the like. For example, the closed-system flow channels 60A to 60C feed liquids such as culture media and cell suspensions from the reagent container 70 to the culture container 80A during a cell culture process such as medium exchange.

One end of the closed-system flow channel 60A is connected to the reagent container 70 in a communicable manner via a connection J1. The other end of the closed-system flow channel 60A is closed so that it does not come into contact with the outside. One end of the closed-system flow channel 60B is connected to the reagent container 70 in a communicable manner via a connection J2. The other end of the closed-system flow channel 60B is connected to the culture container 80A in a communicable manner via a connection J3. One end of the closed-system flow channel 60C is connected to the culture container 80A in a communicable manner via a connection J4. The other end of the closed-system flow channel 60C is closed so that it does not come into contact with the outside.

Thus, the closed-system flow channels 60A to 60C, the reagent container 70, and the culture container 80A constitute a single closed-system flow channel as a whole.

The closed end of the closed-system flow channel 60A has a connection (not shown) that can connect the closed-system flow channel 60A and another closed-system flow channel to each other in a communicable manner. For example, what is called an aseptic connection fitting can be used as the connection. Thus, a single closed-system flow channel can be configured even when, for example, the configuration of the flow channels of the cell culture apparatus 200 is changed as described below. The closed-system flow channel 60C also has the same connection as that of the closed-system flow channel 60A.

The reagent container 70 is a container configured to store reagents such as culture media for use in the cell culture process. The culture container 80A is a container configured to culture the target cells.

The connections J1 to J4 are locations in which liquid leakage can occur due to feeding of reagents such as culture media, samples suspended in cell suspensions, or the like. For example, if there are gaps or looseness in the connections J1 to J4, reagents or samples may leak from the closed-system flow channels. If there are gaps or looseness in the connections J1 to J4, liquid may enter the inside of the closed-system flow channels from the outside.

Thus, from the viewpoint of infection prevention (to prevent leakage of substances from the inside to the outside of the closed-system flow channels) and contamination prevention (to prevent foreign matter from mixing from the outside to the inside of the closed-system flow channels), the detection member reacting to change in properties on contact with liquid is provided to the locations at which liquid leakage can occur, such as the connections J1 to J4, thereby detecting the occurrence of liquid leakage or contamination.

FIG. 6 illustrates an example in which the entire closed-system flow channels and containers are filled with liquid, but the state of the liquid present in the closed-system flow channels and the containers is not limited to this example. For example, liquid may be present only in the containers, and liquid may be present in the closed-system flow channels only during liquid feeding. The same also applied to the other drawings.

By the way, the flow channel configuration of the cell culture apparatus may change during the cell culture process. For example, to collect the cell cultured in the culture container 80A into another container, a separate flow channel or the like may be connected to the flow channel of the cell culture apparatus.

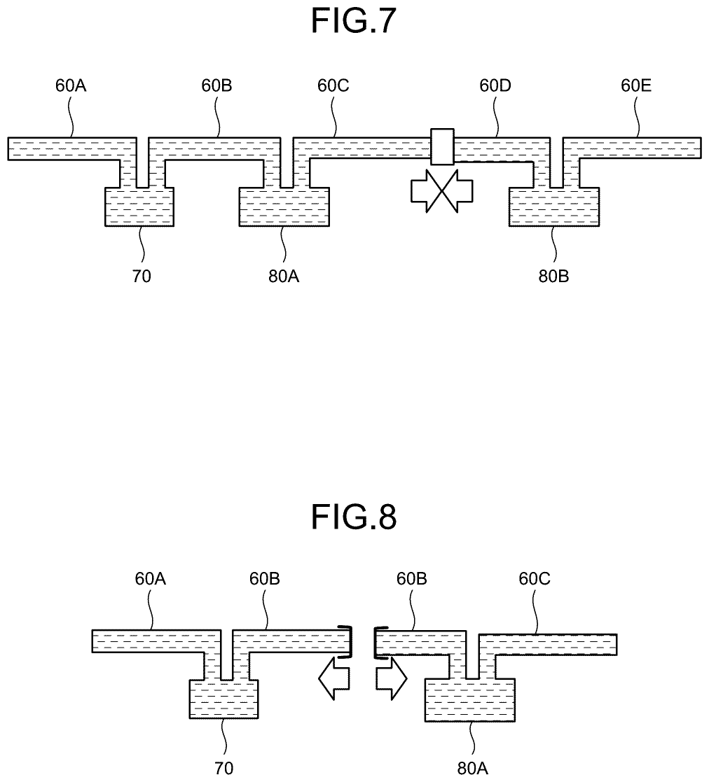

FIG. 7 is an example of a case in which another flow channel is connected to the cell culture apparatus 200. In FIG. 7, another closed-system flow channel 60D is connected to the closed-system flow channel 60C. The closed-system flow channel 60D is connected to a collection container 80B, and the collection container 80B is connected to still another closed-system flow channel 60E.

The end of the closed-system flow channel 60E that is not connected to the collection container 80B is closed so that it does not come into contact with the outside. Thus, even when the closed-system flow channel 60D, the collection container 80B, and the closed-system flow channel 60E are connected to the cell culture apparatus 200, a single closed-system flow channel is formed as a whole in the cell culture apparatus 200.

In this case, the cell culture apparatus 200, for example, feeds a cell suspension from the reagent container 70 to the culture container 80A and collects the target cells in the collection container 80B. For example, by connecting the closed-system flow channel 60E to still another closed-system flow channel, processing such as testing the characteristics of the target cells can be performed.

There may also be a case in which in the cell culture process, after supplying a specific reagent to the culture container, the flow channel is disconnected in order to, for example, avoid the reagent from flowing into the culture container in excess of a required amount.

FIG. 8 is an example of a state in which the flow channel of the cell culture apparatus 200 is disconnected. In FIG. 8, part of the closed-system flow channel 60B is disconnected. This makes it possible, for example, to prevent the supply of the reagent to the target cells in excess of the required amount or to supply the reagent in the reagent container 70 to different cells from the target cells in the culture container 80A by connecting the closed-system flow channel 60B to another culture container or the like after disconnection.

Thus, the configuration of the flow channels of the cell culture apparatus 200 may change during the cell culture process. When the configuration of the flow channels of the cell culture apparatus 200 changes, if locations at which liquid leakage may occur are not identified in advance, it may not be able to detect liquid leakage effectively. In addition, the detection member is required to be provided to the parts at which liquid leakage may occur every time the configuration changes, which is inefficient.

Given these circumstances, the cell culture apparatus of the present embodiment has a configuration with a double structure container in which closed-system flow channels (including containers such as a reagent container and a culture container) are housed in a housing container, with a detection member provided inside the housing container. This configuration enables the cell culture apparatus of the present embodiment to detect liquid leakage no matter where liquid leakage occurs in the closed-system flow channels.

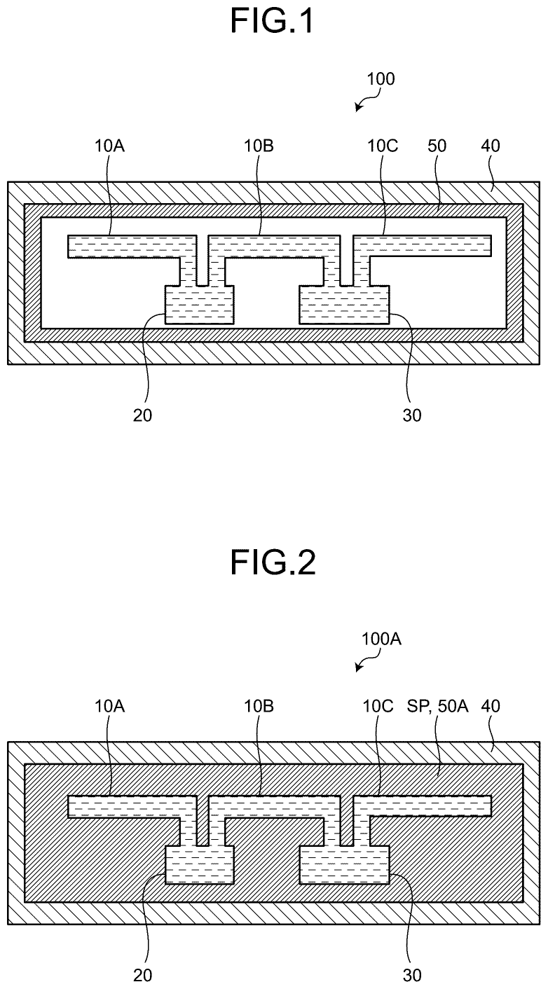

The following describes the details of the cell culture apparatus according to the present embodiment with reference to an example. FIG. 1 is a diagram illustrating an example of a cell culture apparatus 100 according to a first embodiment.

The cell culture apparatus 100 includes closed-system flow channels 10A to 10C, a reagent container 20, a culture container 30, a housing container 40, and a detection member 50. The closed-system flow channels 10A to 10C, the reagent container 20, and the culture container 30 are similar to the closed-system flow channels 60A to 60C, the reagent container 70, and the culture container 80A in FIG. 6, and thus descriptions thereof are omitted. In the following description, the closed-system flow channels 10A to 10C, the reagent container 20, and the culture container 30 are also referred to as a closed-system device.

The housing container 40 is a container housing the closed-system device. By housing the closed-system device in the housing container 40, the cell culture apparatus 100 constitutes a container housing a cell suspension and the like by a double structure of the closed-system device and the housing container 40. The cell culture apparatus 100 may be configured as a multiple structure container by providing the housing container 40 with a double or more complex structure.

In the present embodiment, the housing container 40 is formed of a rigid body such as resin or metal and has a shape that can house the closed-system device of a rectangular parallelepiped shape, a cubic shape, or the like. As illustrated in FIG. 1, a detection space SP is provided between an inner wall face of the housing container 40 and the closed-system device. The housing container 40 is at least partially transparent so that a user can view the detection space SP inside the housing container 40.

In the present embodiment, the housing container 40 is formed transparent on all faces, but only one specific face may be transparent. Only a part of one particular face may be transparent. The transparent part can also be light-blocked as needed. This configuration enables storage of reagents that require light blocking and culture of the target cells in a light-blocked environment.

The housing container 40 may be openable and closable. When the housing container 40 is openable and closable, a gasket or the like is preferably provided at an opening-and-closing part in order to prevent liquid leakage of reagents or samples inside the housing container 40 and entry of foreign matter from outside the housing container 40.

This configuration enables the user to perform manual processing such as taking some of the target cells using a pipette for the target cells. The housing container 40 may also include a double (multiple) structure for each compartment. For example, in FIG. 1, a double structure may be configured separately for each of a compartment including the reagent container 20 and the closed-system flow channels connected to the reagent container 20 and a compartment including the culture container 30 and the closed-system flow channels connected to the culture container 30.

The detection member 50 is a member configured to detect a liquid present in the detection space SP of the housing container 40. As an example, the detection member 50 is a liquid reagent and is applied to the inner wall face of the housing container 40. The detection member 50 is, for example, a liquid reagent the chemical properties of which change on contact with liquid (for example, an aqueous cobalt chloride solution reacting with water to change its color).

The detection member 50 may be applied to the entire inner wall face of the housing container 40 or applied only to a partial area (for example, a bottom face area of the housing container 40).

Since the detection member 50 changes its color on contact with liquid, for example, when liquid leakage occurs from the closed-system device into the detection space SP or when liquid enters the detection space SP from outside the housing container 40, the color changes when the liquid comes into contact with the detection member 50 inside the wall face of the housing container 40. This change enables the user to check whether liquid is present in the detection space SP of the housing container 40 by viewing the inner wall face of the housing container 40.

When the housing container 40 has an openable-and-closable structure, the detection member 50 applied to the inner wall face may be lost due to friction or the like caused by opening and closing. For this reason, the cell culture apparatus 100 may be able to reapply the detection member 50, which is a liquid reagent, to the inner wall face of the housing container 40 using a spray nozzle or the like each time opening and closing is performed.

In the present embodiment, the detection space SP is preferably filled with a gas that does not react with the detection member 50. This configuration can prevent false detection of the presence of liquid due to a change in the chemical properties of the detection member 50 caused by a reaction between the gas present in the detection space SP and the detection member 50.

As described above, the cell culture apparatus 100 according to the first embodiment constitutes the double structure container with the closed-system device and the housing container 40. Inside the housing container 40, the detection member 50 that can detect liquid leakage from the closed-system device is provided.

This configuration makes the closed-system device housed inside the housing container 40 while processing such as cell culture is being performed. In the cell culture apparatus 100, when liquid leaking from the closed-system device or liquid entering from outside the housing container 40 comes into contact with the detection member 50, the presence of the liquid is detected by the detection member 50 visually changing.

Thus, for example, if there is liquid leakage from the closed-system device during processing, the cell culture apparatus 100 according to the present embodiment can detect the liquid leakage no matter where liquid leakage occurs in the closed-system device. Thus, it is no longer necessary to identify in advance the locations at which liquid leakage may occur for the closed-system device. In other words, the cell culture apparatus 100 according to the present embodiment can efficiently detect liquid leakage in the closed-system device.

### Second Embodiment

The first embodiment described above has described a form in which the detection member 50 is applied to the inner wall face of the housing container 40. A second embodiment describes a form of filling the detection space SP inside the housing container 40 with the detection member. In the following description of the second and subsequent embodiments, elements and the like common to those of the first embodiment may be denoted by the same numbers in the drawing, and descriptions thereof may be omitted.

FIG. 2 is a diagram illustrating an example of a cell culture apparatus 100A according to the second embodiment.

The cell culture apparatus 100A includes the closed-system flow channels 10A to 10C, the reagent container 20, the culture container 30, the housing container 40, and a detection member 50A. The closed-system flow channels 10A to 10C, the reagent container 20, the culture container 30, and the housing container 40 are similar to the closed-system flow channels 10A to 10C, the reagent container 20, the culture container 30, and the housing container 40 in FIG. 1, and thus descriptions thereof are omitted.

The detection member 50A is filled in the detection space SP inside the housing container 40. The detection member 50A is, for example, a gas the chemical properties of which change on contact with liquid (for example, a gas the color of which changes on contact with liquid).

Since the detection member 50A changes its color on contact with liquid, for example, when liquid leakage occurs from the closed-system device or when liquid is mixed in from outside the housing container 40, the color of the detection member 50A filled in the detection space SP inside the housing container 40 changes. This change enables the user to check whether liquid is present inside the housing container 40 by viewing the detection space SP inside the housing container 40.

In the present embodiment, even if liquid does not come into contact with the inner wall face of the housing container 40, if liquid is present in the detection space SP, the liquid can be detected, thus making it easier to detect the liquid than a case in which the detection member is applied to the inner wall face of the housing container 40.

When the housing container 40 has an openable-and-closable structure, the detection member 50A filled in the detection space SP may flow outside by the opening and closing. For this reason, the cell culture apparatus 100 may be able to refill the gas detection member 50A in the detection space SP inside the housing container 40 using a gas nozzle or the like each time the opening and closing is performed.

The cell culture apparatus 100A may include a mechanism to detect whether the detection member 50A is filled in a sufficient amount (such as a pressure sensor that can detect the pressure of the detection member 50A). This configuration can prevent detection omission due to an insufficient filling amount of the detection member 50A.

In the present embodiment, the detection member 50A is a gas, but the detection member 50A is not limited to this example. For example, it can be a solid (small particles) or gel-like substance so long as it can be filled in the detection space SP inside the housing container 40.

When the detection member 50A is a solid or gel-like substance, it may be filled in the detection space SP without gaps, or it may be filled only partially in the detection space SP. When it is partially filled, for example, the detection member 50A may be filled in the space SP in such an amount that the closed-system flow channels 10A to 10C, the reagent container 20, and the culture container 30 are covered with the detection member 50A.

As described above, the cell culture apparatus 100A according to the second embodiment includes the detection member 50A filled in the detection space SP inside the housing container 40. This configuration enables detection of liquid present inside the housing container 40 even if the liquid is not into contact with the inner wall face of the housing container 40. Thus, the cell culture apparatus 100A according to the present embodiment can detect liquid present inside the housing container 40 with higher sensitivity.

### Third Embodiment

The first embodiment and the second embodiment described above have described a form in which the user visually check the presence or absence of liquid leakage. A third embodiment describes a form of automatically detecting liquid leakage.

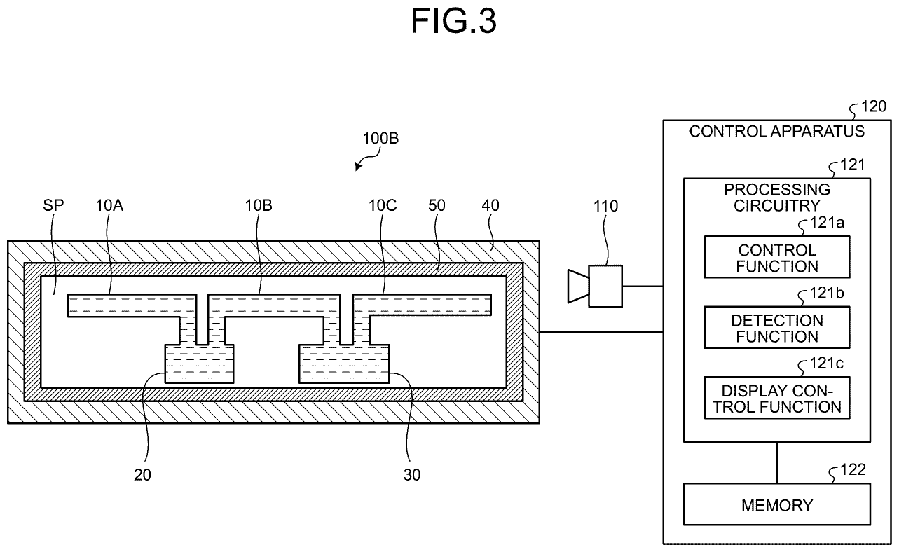

FIG. 3 is a configuration diagram illustrating an example of the configuration of a cell culture apparatus 100B according to the third embodiment. As illustrated in FIG. 3, the cell culture apparatus 100B, for example, has the closed-system flow channels 10A to 10C, the reagent container 20, the culture container 30, the housing container 40, the detection member 50, a camera 110, and a control apparatus 120. The closed-system flow channels 10A to 10C, the reagent container 20, the culture container 30, the housing container 40, and the detection member 50 are similar to those of the first embodiment, and thus descriptions thereof are omitted.

The camera 110 photographs the inside of the housing container 40. The camera 110 is an example of a sensor. For example, the camera 110 is connected to the control apparatus 120. The camera 110 takes camera images of the inside of the housing container 40 under the control of the control apparatus 120. The camera 110 transmits the taken camera images to the control apparatus 120.

In the present embodiment, the camera 110 is provided outside the housing container 40, but the camera 110 may be provided inside the housing container 40. In this case, the camera 110 and the control apparatus 120 may be connected to each other wirelessly or connected in a wired manner. When they are connected to each other in a wired manner, the inside of the housing container 40 is preferably not into contact with the outside of the housing container 40 while the target cells are being cultured.

The control apparatus 120 has processing circuitry 121 and memory 122, which are connected to the camera 110 and the housing container 40. In the control apparatus 120 in FIG. 6, only the processing circuitry 121 and the memory 122 are illustrated, but in reality, the control apparatus 120 also includes an input interface, a display, and the like.

The memory 122 is connected to the processing circuitry 121 and stores therein various data. For example, the memory 122 stores therein the camera images taken by the camera 110 and various computer programs configured to implement various functions by being read and executed by the processing circuitry 121. For example, the memory 122 is implemented by a semiconductor memory element such as a random access memory (RAM) or a flash memory, a hard disk, an optical disk, or the like.

The processing circuitry 121 controls the operation of the cell culture apparatus 100B in accordance with input operations received from an operator via the input interface. For example, the processing circuitry 121 is implemented by a processor.

The processing circuitry 121 reads and executes the computer programs stored by the memory 122 to execute a control function 121a, a detection function 121b, and a display control function 121c.

The control function 121a controls the operation of the entire cell culture apparatus 100B. The control function 121a controls a drive source such as a motor (not shown) to control the opening and closing of the housing container 40. For example, the control function 121a receives camera images from the camera 110. For example, the control function 121a performs control to store the received camera images in the memory 122.

For example, the control function 121a may control a spray nozzle (not shown) to perform, for example, processing to spray a certain amount of the detection member 50 onto the inner wall face of the housing container 40 after the opening and closing of the housing container 40 is performed.

The detection function 121b detects liquid inside the housing container 40 based on the camera images obtained by photographing the inside of the housing container 40. For example, the detection function 121b analyzes the camera images stored in the memory 122 to detect the presence of the liquid inside the housing container 40 from a visual change in the detection member 50 provided to the inner wall face of the housing container 40. Known image analysis techniques can be used for analysis processing on the camera images.

This enables the cell culture apparatus 100B to detect liquid leakage even when liquid leakage occurs from the closed-system device in a situation in which the user is not viewing the housing container 40.

In the present embodiment, the detection function 121b detects the liquid inside the housing container 40 from the visual change in the detection member 50 provided to the inner wall face of the housing container 40, but the method of liquid detection is not limited to this example. For example, the detection function 121b may detect the liquid inside the housing container 40 from a visual change in a gas filled inside the housing container 40.

The display control function 121c performs control to display various information on a display apparatus. For example, the display control function 121c provides notification of a warning on a display (not shown) or the like indicating that liquid leakage may have occurred from the closed-system device when the detection function 121b detects the liquid inside the housing container 40.

In the present embodiment, notification is provided to the user by displaying a warning message, but the processing circuitry 121 may also provide notification to the user by generating a warning sound from a speaker or the like.

As described above, the cell culture apparatus 100B according to the third embodiment detects the liquid inside the housing container 40 based on the camera images taken by the camera 110 photographing the inside of the housing container 40. This enables, for example, the cell culture apparatus 100B according to the present embodiment to detect liquid leakage from the closed-system device even in a situation in which the user cannot view the inside of the housing container 40.

By providing the camera 110 inside the housing container 40, the cell culture apparatus 100B according to the present embodiment can store reagents requiring light blocking in the reagent container 20 and culture the target cells in a light-blocked environment.

The embodiments described above can also be performed in a modified manner as appropriate by changing part of the configurations or functions of each apparatus. Thus, in the following, a modification according to the embodiments described above is described as another embodiment. The following mainly describes points that differ from the embodiments described above and omits detailed descriptions of points common to the details already described. The modification described below may be performed individually or performed in combination with the embodiments described above as appropriate.

### Modification

The first embodiment to the third embodiment described above have described a form of detecting the liquid inside the housing container using the detection member the chemical properties of which change on contact with the liquid. However, the liquid inside the housing container may be detected using the detection member the physical properties of which change on contact with the liquid.

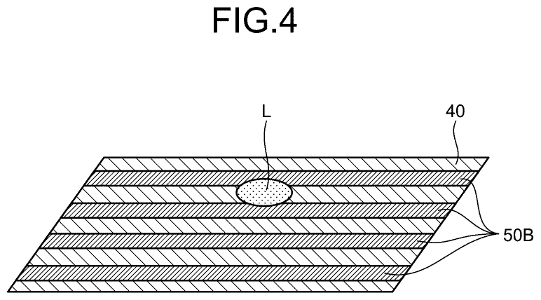

FIG. 4 is a diagram illustrating an example of liquid detection processing according to a modification. The housing container 40 according to the modification includes detection members 50B on the inner wall face. The detection members 50B are a plurality of lead wires disposed on the inner surface of the housing container 40. Although indication in the drawing is omitted, the cell culture apparatus 100 according to the present modification has a voltage application apparatus applying voltage to the detection members 50B. For example, the voltage application apparatus continuously applies voltage to the detection members 50B during the culture of the target cells.

As illustrated in FIG. 4, when a liquid L comes into contact with the detection members 50B, the electrical resistivity changes, and thus the magnitude of a current passing through the detection members 50B changes. Thus, by measuring the magnitude of the current passing through the detection members 50B with a current sensor (not shown), the cell culture apparatus 100 according to the modification can detect the liquid inside the housing container 40. In this case, the current sensor is an example of the sensor.

The cell culture apparatus 100 according to the present modification includes the control apparatus 120 as in the third embodiment. In the present modification, the detection function 121b of the processing circuitry 121 of the control apparatus 120 detects the liquid inside the housing container 40 based on a measurement result of the current sensor. For example, the detection function 121b detects the presence of the liquid inside the housing container 40 when the measured value of the current is not in a certain range.

The method of liquid detection using the current sensor is not limited to the above. For example, a buzzer may be connected to the current sensor, and the buzzer may be sounded when the measured value of the current is not in the certain range.

When the current sensor is used, the housing container 40 does not necessarily have a transparent part. By making the entire housing container 40 opaque, it becomes possible, for example, to store reagents the properties of which are changed by light in the reagent container 70.

In addition, the occurrence of a gap in the detection members 50B may be detected during the opening and closing of the housing container 40 using a current sensor. In this case, the detection members 50B are provided at the opening-and-closing part of the housing container 40. The detection members 50B in this case detect that the housing container 40 is in an open state, not liquid leakage or liquid entry.

For example, by detecting that the housing container 40 is in an open state and providing notification of the fact to the user, the user can quickly perceive the possibility of a sample or reagent leaking from the closed-system flow channels inside the housing container 40 or foreign matter entering the inside of the housing container 40 from outside.

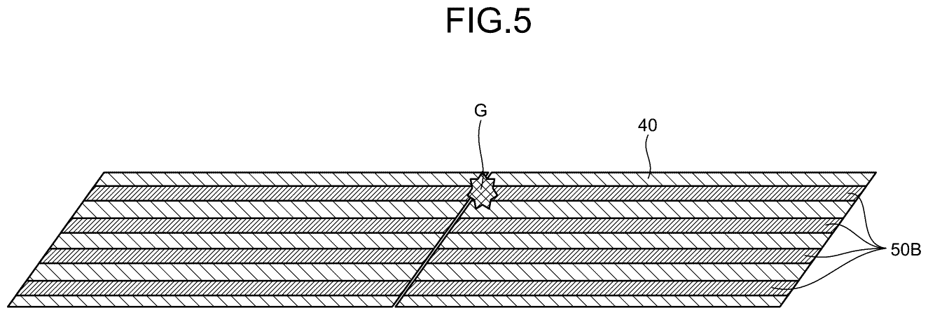

FIG. 5 is a diagram illustrating an example of gap detection processing for the detection members 50B according to the modification. As illustrated in FIG. 5, when a gap G occurs in the detection members 50B, a current becomes not to flow (or becomes difficult to flow) in the detection members 50B.

Thus, the gap G can be detected by measuring the current passing through the detection members 50B with a current sensor during the opening and closing of the housing container 40. In the example in FIG. 5, the detection function 121b detects the gap G of the detection members 50B when the measured value of the current sensor is below a certain threshold.

The cell culture apparatus 100 according to the present modification can detect the liquid inside the housing container 40 without having to perform processing such as reapplication or refilling of the detection member.

The embodiments and the modification described above have described each processing function of the processing circuitry 121. For example, each processing function described above is stored in the memory 122 in the form of a computer program executable by a computer. The processing circuitry 121 reads each computer program from the memory 122 and executes each read computer program to implement the processing function corresponding to each computer program. In other words, the processing circuitry 121 having read each computer program has each processing function illustrated in FIG. 3.

In FIG. 3, an example of a case in which each processing function is implemented by the single processing circuitry 121 has been described, but embodiments are not limited to this example. For example, the processing circuitry 121 may include a plurality of independent processors combined, and each processor may execute each computer program to implement each processing function. Each processing function of the processing circuitry 121 may be implemented distributed or integrated into a single piece of or a plurality of pieces of processing circuitry as appropriate.

The term “processor” used in the description of the embodiments described above means, for example, circuitry such as a central processing unit (CPU), a graphics processing unit (GPU), an application specific integrated circuit (ASIC), and a programmable logic device (for example, a simple programmable logic device (SPLD), a complex programmable logic device (CPLD), and a field programmable gate array (FPGA)).

Instead of storing the computer program in memory, the computer program may be incorporated directly into the circuitry of the processor. In this case, the processor reads and executes the computer program incorporated in the circuitry to implement the function. Each processor in the present embodiment is not limited to a case in which each processor is configured as a single piece of circuitry but may also be configured as one processor by combining a plurality of independent pieces of circuitry to implement its function.

According to at least one of the embodiments described above, liquid leakage in the closed-system devices can be efficiently detected.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A sample processing apparatus comprising:
a closed-system flow channel configured to process a liquid containing a sample;
a housing container housing the flow channel; and
a detection member provided inside the housing container and configured to detect a liquid present in a space inside the housing container.

2. The sample processing apparatus according to claim 1, wherein the detection member is provided to an inner wall face of the housing container.

3. The sample processing apparatus according to claim 1, wherein the detection member is filled in the space inside the housing container.

4. The sample processing apparatus according to claim 1, wherein the detection member is a member changing in chemical or physical properties on contact with liquid, the sample processing apparatus further comprising;
a sensor configured to sense a state of the detection member inside the housing container; and
processing circuitry configured to detect the liquid present in the space inside the housing container based on a sensing result of the sensor and, when the liquid is detected, provide notification of the being detected.

5. The sample processing apparatus according to claim 4, wherein the sensor is provided inside the housing container.

6. The sample processing apparatus according to claim 4, wherein the sensor is a camera configured to photograph the detection member inside the housing container.

7. The sample processing apparatus according to claim 4, wherein
the detection member is a lead wire,
the sensor is a current sensor configured to measure a current passing through the detection member; and
the processing circuitry detects the liquid present in the space inside the housing container based on a measured value of the current.

8. The sample processing apparatus according to claim 1, wherein the detection member contains a substance changing in chemical properties on contact with liquid.

9. The sample processing apparatus according to claim 8, wherein the detection member contains a substance changing its color by the change in chemical properties.

10. The sample processing apparatus according to claim 1, wherein the detection member is a liquid member, the sample processing apparatus further comprising a spray configured to spray and apply the detection member to an inner wall face of the housing container.

11. The sample processing apparatus according to claim 1, wherein a gas non-reacting with the detection member is filled in a detection space that is a space between an inner wall face of the housing container and the flow channel.

12. The sample processing apparatus according to claim 11, wherein the detection member is a gas, small-particle, or gel-like substance, and the detection member is filled in the detection space.

13. The sample processing apparatus according to claim 12, wherein the detection member is filled in the detection space with no gap.

14. The sample processing apparatus according to claim 12, wherein the detection member is a small-particle or gel-like substance, and the detection member is filled in the detection space to cover at least the flow channel.

15. The sample processing apparatus according to claim 1, wherein the housing container is formed of a rigid body.

16. The sample processing apparatus according to claim 15, wherein the housing container is formed of a resin or a metal.

17. The sample processing apparatus according to claim 1, wherein the housing container is at least partially formed of a transparent member.

18. The sample processing apparatus according to claim 17, wherein the housing container has a light-blocker configured to block the part formed of the transparent member from light.

19. The sample processing apparatus according to claim 1, wherein the flow channel includes
a reagent container configured to store a reagent used in a cell culture process, and
a culture container configured to culture a cell.

20. A housing container comprising:
a housing part housing a closed-system flow channel configured to process a liquid containing a sample; and
a detection member provided inside the housing part and configured to detect a liquid present in a space inside the housing part.
